SEQUENCE LISTING (1 sequence)

SEQ ID NO: 1 - protein, 317 aa, Clostridium chauvoei
MIKRILMLALATTTIFSLTLPFSYKAVQAQENTCIVETPSEGVKTFTSSDTAYADYNCFKTNLSVTFIEDQHNNQLTALVSTEGSFIPSGLSRVGGYYQADMYWPSKYYTTLTTYDRNNRVKITKSIPTNQIDTVSVSETMGYSIGGSLSIEYGKEGPKAGGGINGSYTAQRSVTYDQPDYRTLLMKDSVNSASWEVAFNATKDGYDRDSYHGIYGNQLFMRYRLYNTGINNLTTDNNLSSLIVGGFSPKVVIALTAPKGTEESTVKVEYNRFNDQYRLRWSGTEWYGENNRNSRIDSSSESFILNWKNHTVEHAGY

(19) United States
(12) Patent Application Publication
(10) Pub. No.: US 2025/0099565 A1
(43) Pub. Date: Mar. 27, 2025

(54) CLOSTRIDIUM CHAUVOEI VACCINE AND METHOD OF MAKING

(71) Applicant: Zoetis Services LLC, Parsippany, NJ (US)

(72) Inventor: Anthony James CAMERON, Melbourne (AU)

(21) Appl. No.: 18/293,539

(22) PCT Filed: Aug. 5, 2022
(86) PCT No.: PCT/US2022/074574

§ 371 Date: Jan. 30, 2024

Related U.S. Application Data
(60) Provisional application No. 63/230,176, filed on Aug. 6, 2021.

(51) Int. Cl. A61K 39/08; A61K 39/00; A61K 39/02; C07K 1/34; C12N 1/20

(52) U.S. Cl. CPC A61K 39/08; A61K 39/0225; C07K 1/34; C12N 1/20; A61K 2039/521; A61K 2039/552; A61K 2039/55505; A61K 2039/70

(57) ABSTRACT

The invention provides a vaccine against C. chauvoei, the vaccine comprising a C. chauvoei component and additional cctA protein. The methods of making and using said vaccine are also provided.

### FIELD OF THE INVENTION

This invention is in the field of clostridial vaccines for ruminants.

### BACKGROUND

Clostridial myositis (blackleg) caused by Clostridium chauvoei is an extremely economically important disease of sheep and cattle. Clostridium chauvoei is a spore-forming, Gram-positive anaerobic bacillus. Its spores are ubiquitous in the soil and manure, and after ingestion they are capable of crossing the intestinal mucosa, entering the bloodstream, and being carried to skeletal muscles. The spores lie dormant until localized trauma to the muscle, which in cattle is most often caused by bruising during handling in a chute or from trauma in a crowded feedlot, results in muscle damage and localized hypoxia and anoxia.

Current knowledge on pathogenicity of C. chauvoei reveals that toxins, a highly active DNAse, hyaluronidase, sialidase, and flagella represent the main virulence factors. Among the postulated toxins, C. chauvoei toxin A (CctA) was shown to represent the main cytotoxic and haemolytic activity of C. chauvoei.

Besides toxin CctA, sialidase NanA and hyaluronidase NagH, seem to enable C. chauvoei to migrate from the initial site of infection which are generally believed to be oral or the respiratory tract or occasionally skin lesions, and get access to muscular tissue where the bacterium can replicate and cause myonecrosis.

There is generally no effective treatment for cattle with blackleg, and death occurs rapidly. Prevention is the best approach. Vaccination against clostridial toxins and maintenance of a safe environment are critical.

Vaccines against blackleg consist of chemically inactivated bacteria providing outer membrane proteins and flagellar proteins that have been proposed as immunogens and bacterial culture supernatants that are expected to contain the main toxins. Recently it has been shown that the CctA toxin alone, which is conserved between C. chauvoei strains isolated worldwide, prepared by recombinant gene technology, provided effective protection in a guinea pig infection model which serves as biological test for potency tests in the batch release procedure of commercial vaccines.

Although the literature on blackleg is voluminous, scientific evidence on the efficacy of vaccination against C. chauvoei to prevent diseases and lethality in cattle is scant. Uzal et al acknowledged that the evidence of efficacy of C. chauvoei vaccines to prevent infection by this microorganism in cattle is poor to moderate. See Vet Clin North Am Food Anim Pract. 2012 March; 28 (1): 71-7, viii. Accordingly, there is need in the art for additional vaccines capable of protecting ruminants against C. chauvoei infections.

### SUMMARY OF INVENTION

In the first aspect, the disclosure provides a vaccine against Clostridium chauvoei, said vaccine comprising a C. chauvoei component and additional cctA protein.

In different embodiments, of this first aspect, one dose of said vaccine contains at least about 3.5 μg of said additional cctA protein, and 0.025 to 0.148 RU of flagella. In certain preferred embodiments, the vaccine according to the first aspect of the invention contains about 0.074 RU of said flagella and about 5 μg of said additional cctA protein. In the vaccine according to any embodiments of the first aspect, the cctA protein is produced from the supernatant of cultured C. chauvoei and/or the C. chauvoei component is a bacterin, a whole cell extract, a partial cell extract, or a combination thereof.

In the second aspect, the disclosure provides a method preparing a vaccine against C. chauvoei infection, said method comprising:
a. Culturing C. chauvoei;
b. Collecting culture media;
c. Concentrating C. chauvoei from said culture media;
d. Concentrating cctA from said culture media; and
e. Combining said concentrated cctA with C. chauvoei.

In certain embodiments, the method comprises a step of microfiltrating said culture media to obtain a microfiltered retentate and a microfiltered permeate.

In certain embodiments according to this second aspect of the invention, the step of culturing C. chauvoei may be performed in a culture media comprising a peptone of plant origin, a peptone of animal origin, or a combination thereof.

In certain embodiments, the step of concentrating said cctA from said culture media comprises:
a. Ultrafiltration of said microfiltered permeate, to obtain an ultrafiltered retentate, followed by
b. Diafiltration, to obtain a diafiltered retentate, followed by
c. Sterile filtration, to obtain a sterile solution of said additional cctA protein.

In certain embodiments, the ultrafiltration comprises passing said microfiltered permeate through a 10 kDa cassette. In certain embodiments, the diafiltration comprises passing said ultrafiltered retentate through a 10 kDa cassette and/or the diafiltration is followed by a sterile filtration of the diafiltered retentate through a 0.2 μm filter to prepare the sterile solution of additional cctA protein.

Preferably, in the method according to any of the embodiments of this second aspect of the invention, the microfiltered retentate is diafiltered to obtain the C. chauvoei component.

In the most preferred embodiment, at least a portion of the C. chauvoei component and at least a portion of the sterile solution of additional cctA protein are combined.

In the third aspect, the disclosure provides a vaccine prepared according to any embodiments of the second aspect of the invention wherein one dose of said vaccine contains at least 3.5 μg of said concentrated cctA protein.

In certain embodiments of the third aspect, one dose of the vaccine contains about 0.074 to 0.148 RU of said flagella. Preferably, one dose contains about 5 μg of said concentrated cctA and even more preferably, one dose contains about 5 μg of said concentrated cctA and about 0.074 Ru of said flagella.

In certain embodiments, the vaccine may contain at least one additional antigen selected from the group consisting of Clostridium septicum, Clostridium haemolyticum, Clostridium novyi, Clostridium sordellii, Clostridium tetani, Clostridium perfringens types C and D, Erysipelothrix rhusiopathiae, Leptospira borgpetersenii serovar Hardjo, Leptospira interrogans serovar Pomona and Mannheimia haemolytica antigens. In certain embodiments, said at least one additional antigen is an inactivated culture or toxoid. In any embodiments of this third aspect, the vaccine may be non-adjuvanted or may comprise an adjuvant.

In the fourth aspect, the disclosure provides a method of preventing C. chauvoei infection in a subject comprising administering to said subject the vaccine according to any one of the embodiments of the first or the third aspect of the invention. In certain embodiments of this fourth aspect, said subject is a bovine or an ovine subject.

### BRIEF DESCRIPTION OF THE DRAWINGS

FIG. 1 is a schematic illustration of a process of preparing the C. chauvoei component and the concentrated cctA for the preparation of the vaccine according to certain embodiments of the invention.

### DETAILED DESCRIPTION

For the better understanding of the invention, the following non-limiting definitions are provided:

The phrase “a vaccine comprising additional cctA protein” refers to a vaccine containing a C. chauvoei component spiked with additional purified or a partially purified cctA protein. Thus, in the vaccine comprising additional cctA protein, the ratio of cctA to flagella (a proxy for the amount of C. chauvoei) will be greater than said ratio in a vaccine lacking this additional cctA. In certain embodiments, the ratio of cctA to flagella is at least 50% greater, or at least 100% greater, or at least 300% greater or at least 500% greater or at least 1000% greater or at least 1500% greater or at least 2000% greater.

The term “about” as applied to a reference number refers to the reference number plus or minus 10 percent of said value.

The term “bacterin” refers to a suspension of killed bacteria.

The term “C. chauvoei component” refers to C. chauvoei bacterin, C. chauvoei whole cell extract, C. chauvoei partial cell extract, or any combination thereof.

The term “culture”, as used herein, means a population of cells or microorganisms growing in the absence of other species or types.

“Dose” refers to a vaccine or immunogenic composition given to a subject. A “first dose” or “priming dose” refers to the dose of such a composition given on Day 0. A “second dose” or a “third dose” or an “annual dose” refers to an amount of such composition given subsequent to the first dose, which can be but is not required to be the same vaccine or immunogenic composition as the first dose.

“Lysate” refers to the product of lysis which is the disintegration of a cell by rupture of the cell wall or membrane. The disintegration may be achieved by mechanical or chemical means known in the art.

The term “partial cell extract” refers to a fraction of the whole cell extract, wherein the extract is able to elicit immune response which is about the same as the immune response elicited by the whole cell extract. Preferably, the partial cell extract comprises cell wall and/or cell membrane fraction. For the avoidance of the doubt, partial cell extract does not include purified and/or concentrated cctA.

“Protection”, “protecting”, “protective immunity”, and the like, as used herein with respect to a vaccine or other composition, means that the vaccine or composition prevents or reduces the symptoms of the disease caused by the organism from which the antigen(s) used in the vaccine or composition is derived. The terms “protection”, “protecting”, and the like, also mean that the vaccine or composition can be used to “treat” the disease, or one or more symptoms of the disease that already exists in a subject.

“Therapeutically effective amount” refers to an amount of an antigen or vaccine that would induce an immune response in a subject receiving the antigen or vaccine which is adequate to prevent or reduce signs or symptoms of disease, including adverse health effects or complications thereof, caused by infection with a pathogen, such as a virus or a bacterium. Humoral immunity or cell-mediated immunity or both humoral and cell-mediated immunity may be induced. The immunogenic response of an animal to a vaccine may be evaluated, e.g., indirectly through measurement of antibody titers, lymphocyte proliferation assays, or directly through monitoring signs and symptoms after challenge with wild type strain. The protective immunity conferred by a vaccine can be evaluated by measuring, e.g., reduction in clinical signs such as mortality, morbidity, temperature number, overall physical condition, and overall health and performance of the subject. The amount of a vaccine that is therapeutically effective may vary depending on the particular adjuvant used, the particular antigen used, or the condition of the subject, and can be determined by one skilled in the art.

“Treating” refers to preventing a disorder, condition, or disease to which such term applies, or to preventing or reducing one or more symptoms of such disorder, condition, or disease.

“Vaccine” or “vaccine composition,” as used herein, refers to an immunogenic composition containing the antigens and capable of eliciting specific immune response against the antigen. Administration of the vaccine to a subject results in a protective immune response, which can be completely protective or partially protective. The vaccine can be introduced directly into the subject by any known route of administration, including parenterally, perorally, and the like. The terms mean a composition which prevents or reduces an infection, or which prevents or reduces one or more signs or symptoms of infection. The protective effects of a vaccine composition against a pathogen are normally achieved by inducing in the subject an immune response. Generally speaking, abolished or reduced incidences of infection, amelioration of the signs or symptoms, or accelerated elimination of the microorganism from the infected subjects are indicative of the protective effects of a vaccine composition.

“Veterinarily acceptable”, as used herein, refers to substances which are, within the scope of sound medical judgment, suitable for use in contact with the tissues of veterinary subjects without undue toxicity, irritation, allergic response, and the like, commensurate with a reasonable benefit-to-risk ratio, and effective for their intended use.

“Veterinarily acceptable carrier”, as used herein, refers to a carrier medium that does not interfere with the effectiveness of the biological activity of the active ingredient, and is not toxic to the veterinary subject to whom it is administered.

The term “whole cell extract” or “whole organism extract” and the like refers to a cell lysate, including bacterial cell lysate, e.g., C. chauvoei cell lysate.

The present disclosure is based on unexpected discoveries that it is possible to concentrate cctA from the supernatant of cultured C. chauvoei, that addition of this concentrated cctA to C. chauvoei component provides a more efficient vaccine that C. chauvoei alone and that the amount of C. chauvoei component per dose can be reduced without sacrifice in the efficiency of the vaccine if the vaccine contains additional cctA protein.

Accordingly, in the first aspect, the invention provides a vaccine comprising a Clostridium chauvoei component and further comprising additional cctA protein.

C. chauvoei Component

In certain embodiments, C. chauvoei compound is a bacterin. In other embodiments, C. chauvoei is a whole cell extract. In yet other embodiments, C. chauvoei is a partial cell extract. Preferably, the partial cell extract comprises C. chauvoei membrane fraction and/or C. chauvoei cell wall fraction. Different combinations of these embodiments are also possible.

According to the disclosure, the amount of C. chauveoei component is quantified using relative units (RU) of flagella. Thus, the use of flagella RU is a proxy for the amount of C. chauvoei. It should be understood, however, that other protein(s) can be used as a proxy for quantification of the amount of C. chauveoei component.

Multiple methods are known for preparing C. chauvoei component. For example, cultured and harvested C. chauvoei may be inactivated with formalin, betapropriolactone (BPL) or binary ethyleneimine (BEI), or other methods known to those skilled in the art.

Whole cell extracts can be prepared by rupturing cell wall and cell membrane of the bacteria. This can be achieved by many means, including chemical and physical means. For example, the bacteria may be lysed by using hypotonic buffer and/or lyophilization and/or centrifugation. Partial cell extracts may be obtained, for example, by ultracentrifugation of C. chauvoei preparation.

Non-limiting examples of suitable preparations of C. chauvoei include products that are currently on the market as a component of commercially available vaccines, including, without limitations ONE SHOT ULTRA® 8 or ULTRAVAC® 7 IN 1 by Zoetis.

In certain embodiments, C. chauvoei component is a bacterin prepared by culturing C. chauvoei, followed by inactivation with formalin. The suspension containing inactivated C. chauvoei is then microfiltered through 0.2 μm cassette, and then diafiltered with saline through a 0.2 μm microfiltration cassettes.

Additional cctA Protein

CctA protein according to the invention comprises SEQ ID NO: 1:

| | |
|---|---|
| 1 | mikrilmlal atttifsltl pfsykavqaq entcivetps egvktftssd tayadyncfk |
| 61 | tnlsvtfied qhnnqltalv stegsfipsg lsrvggyyqa dmywpskyyt tlttydrnnr |
| 121 | vkitksiptn qidtvsvset mgysiggsls ieygkegpka gggingsyta qrsvtydqpd |
| 181 | yrtllmkdsv nsaswevafn atkdgydrds yhgiygnqlf mryrlyntgi nnlttdnnls |
| 241 | slivggfspk vvialtapkg teestvkvey nrfndqyrlr wsgtewygen nrnsridsss |
| 301 | esfilnwknh tvehagy |

or a protein that is at least 90% identical to SEQ ID NO: 1, and may be at least 91%, or at least 91%, or at least 92%, or at least 93%, or at least 94%, or at least 95%, or at least 96%, or at least 97%, or at least 98%, or at least 99% identical to SEQ ID NO: 1. Preferably, amino acids differing between the sequence of cctA and SEQ ID NO: 1 are substitutions, and more preferably, at least 50%, or at least 60% or at least 70% or at least 80% or at least 90% or at least 95% or all 100% of these substitutions are conservative substitutions.

The skilled person will recognize that alterations of the nucleic acid sequence resulting in modifications of the amino acid sequence of the protein it codes may have little, if any, effect on the resulting three-dimensional structure of the protein. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in the substitution of one negatively charged residue for another, such as aspartic acid for glutamic acid, or one positively charged residue for another, such as lysine for arginine, can also be expected to produce a protein with substantially the same functional activity.

The following six groups each contain amino acids that are typical conservative substitutions for one another: [1] Alanine (A), Serine(S), Threonine (T); [2] Aspartic acid (D), Glutamic acid (E); [3] Asparagine (N), Glutamine (Q); [4] Arginine (R), Lysine (K), Histidine (H); [5] Isoleucine (I), Leucine (L), Methionine (M), Valine (V); and [6] Phenylalanine (F), Tyrosine (Y), Tryptophan (W), (see, e.g., US Patent Publication 20100291549).

C. chauvoei cctA protein may be prepared by multiple methods, including purification of cctA proteins from the supernatant of the cultured C. chauvoei and/or genetic engineering and/or chemical synthesis. These methods are well known in the art.

For the purposes of this disclosure, the amount of C. chauvoei component in one dose of a vaccine is determined by the amount of flagella, measured in Relative Units, or RUs. A single dose of a vaccine without additional cctA contains about 0.148 RU of flagella. It should be noted however, that other methods for determining the amount of C. chauvoei compound do exist and it is possible to convert the end results of these methods into flagella RUs.

In the vaccines disclosed herein and comprising additional cctA protein, the ratio of cctA to flagella will be greater than said ratio in a vaccine lacking this additional cctA. In certain embodiments, the ratio of cctA to flagella is at least 50% greater, preferably, at least 75% greater, at least 100% greater, at least 150% greater, at least 200% greater, at least 250% greater, at least 300% greater, at least 350% greater and so on compared to a composition that comprises the C. chauvoei component but lacks the added cctA protein.

In certain embodiments, the vaccine according to the invention contains about 3.5 μg of said additional cctA protein per dose or more, and may contain up to 30 μg of said additional cctA protein per dose and in different embodiments, the vaccine may contain 3.5 to about 4 μg of said additional cctA protein per dose, or 3.5 to about 6 μg of said additional cctA protein per dose or 3.5 to about 9 μg of said additional cctA protein per dose or 3.5 to 10 μg of said additional cctA protein per dose or 3.5 to about 15 μg of said additional cctA protein per dose or 3.5 to about 20 μg of said additional cctA protein per dose or 3.5 to about 25 μg of said additional cctA protein per dose or 3.5 to about 30 μg of said additional cctA protein per dose or about 4 to about 30 μg of said additional cctA protein per dose or about 4 to about 25 μg of said additional cctA protein per dose or about 4 to about 20 μg of said additional cctA protein per dose or about 4 to about 15 μg of said additional cctA protein per dose or about 4 to about 10 μg of said additional cctA protein per dose or about 4 to about 8 μg of said additional cctA protein per dose or about 4 to about 6 μg of said additional cctA protein per dose or about 5 μg of said additional cctA protein per dose.

Addition of cctA to the C. chauvoei component allows to lower the dose of the component from about 0.148 RU of flagella to about 0.1 RU of flagella or even lower, e.g., to about 0.074 RU of flagella, or to about 0.05 RU of flagella.

It would be understood that higher amounts of additional cctA would allow for lower amounts of C. chauvoei components. In practical terms, addition of 0.1 μg of cctA would result in the possibility of lowering the amount of C. chauvoei component by about 0.00175 RU of flagella. Preferably, the amount of flagella is no less than about 0.025 RU per dose, more preferably no less than 0.055 RU per dose, and more preferably, no less than 0.060 RU, or no less than 0.65 RU or no less than 0.070 RU, or no less than 0.074 RU per dose.

### Method of Making the Vaccine

In the second aspect, the disclosure provides a method of method of preparing a vaccine against C. chauvoei infection. Generally, when C. chauvoei are cultured for the manufacturing of the vaccine, cctA-containing supernatant is discarded. However, the inventors have discovered that the vaccine may be made by a method which utilizes concentrating cctA from the supernatant, said method comprising:
a. Culturing C. chauvoei;
b. Collecting culture media;
c. Concentrating C. chauvoei from said culture media
d. Concentrating cctA from said culture media; and
e. Combining said concentrated cctA with C. chauvoei.

In certain embodiments, the C. chauvoei are grown in a media containing peptone, which is a product of protein hydrolysis. In certain embodiments, the peptone may be of plant origin (e.g., soy peptone) or animal origin (e.g., meat peptone or casein peptone) or any combination thereof. In a set of preferred embodiments, the peptone is soy peptone.

In different embodiments, the concentration of the peptone in the culture medium is about 20 g/L to about 100 g/L, e.g, about 30 g/L or 40 g/L, or about 50 g/L, or about 60 g/L, or about 70 g/L or about 80 g/L or about 90 g/L. In certain embodiments, the concentration is about 25 to about 75 g/L, and more preferably about 45 to about 55 g/L.

C. chauvoei may be concentrated by microfiltration followed by diafiltration. In certain embodiments, the microfiltration is accomplished by passing the solution through 100,000 kDa to 0.45 μm cassettes and subsequently diafiltered through 100,000 kDa to 0.45 μm cassettes to prepare the C. chauvoei component. For example, the cassettes used for the microfiltration and the diafiltration steps may be independently 0.1, 0.15, 0.2, 0.25, 0.3, 0.35, 0.4 or 0.45 μm. In certain embodiments, both the microfiltration and the diafiltration cassettes are 0.2 μm.

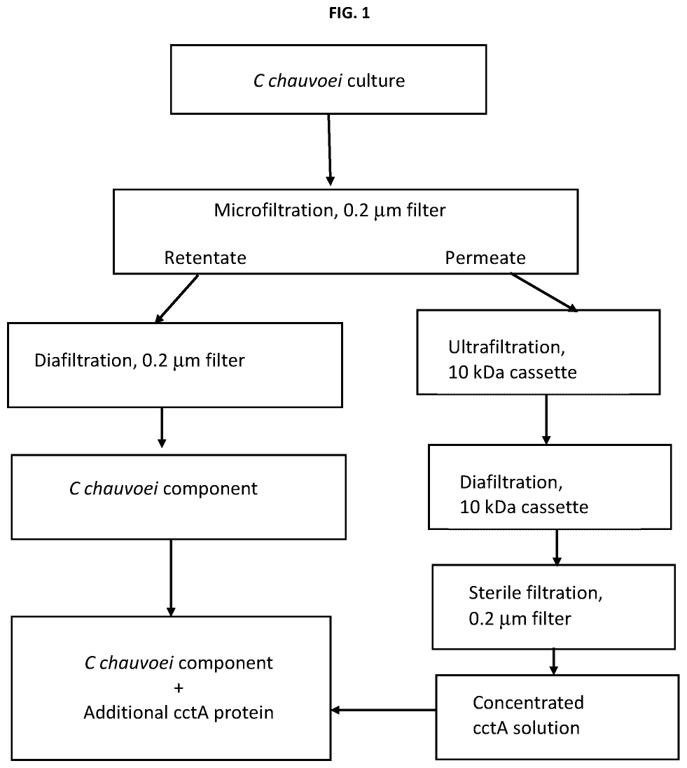

Advantageously, the method of preparing the C. chouvoei component and the method of preparing the concentrated (or enriched) cctA solution may start with common step of microfiltration, preferably through the 0.2 μm cassette. After the step of microfiltration, the microfiltered retentate is diafiltered and the C. chauvoei component is prepared. The microfiltered permeate is then ultrafiltered through a cassette, to obtain the ultrafiltered retentate, and then the ultrafiltered retentate is diafiltered as described above to obtain the diafiltered retentate, and cctA solution is thus prepared. See FIG. 1.

The size of cctA is about 32 kDa. Accordingly, the cassettes used to concentrate cctA should be less than 32 kDa. In certain embodiments, the cassettes are 30 kDa, 20 kDa, 10 kDa, or 5 kDa. In the method according to the invention it is preferable if 10 kDa cassettes are used in this step.

In certain embodiments, the step of concentrating said cctA from said culture media comprises ultrafiltration, to obtain an ultrafiltered retentate, followed by diafiltration, to obtain a diafiltered retentate, followed by sterile filtration, to obtain a sterile solution. These procedures are known in the art.

Ultrafiltration is a variety of membrane filtration in which forces like pressure or concentration gradients lead to a separation through a semipermeable membrane. Suspended solids and solutes of high molecular weight are retained (retentate) while water and low molecular weight solutes pass through the membrane (permeate). This particular process is used to purify and concentrate compounds having molecular weight from 10³ to 10⁶ Da, particularly proteins. Ultrafiltration is generally defined by the molecular weight cut-off of the membrane used in the process.

Diafiltration is a specialized type of ultrafiltration process in which the retentate is diluted with a buffer such as saline, PBS, or water and re-ultrafiltered, to reduce the concentration of soluble permeate components and maintain the concentration of retained components. Cassettes of the same size, such as, for example, 10 kDa, may be used for both ultrafiltration and diafiltration.

In certain preferred embodiments, after the steps of ultrafiltration and diafiltration, the concentrated cctA is sterile filtered. The sterile filtered cctA solution can be added to the C. chauvoei component to prepare the vaccine or immunogenic composition disclosed herein.

### The Vaccines and Immunogenic Compositions

The vaccines and immunogenic compositions according to the invention may contain other antigens, in addition to the combination of the C. chauvoei component and the additional cctA protein. Particularly preferred antigens are derived from pathogens affecting ruminants. In certain embodiments, the antigens may be derived from selected from the group consisting of Clostridium septicum, Clostridium haemolyticum, Clostridium novyi, Clostridium sordellii, Clostridium tetani, Clostridium perfringens types C and D, Erysipelothrix rhusiopathiae, Leptospira borgpetersenii serovar Hardjo, Leptospira interrogans serovar Pomona and Mannheimia haemolytica antigens.

Preferably, these antigens are inactivated organisms, or attenuated organism, or bacterin toxoids, or whole organism extracts or partial organism extracts.

The vaccines and the immunogenic compositions may also contain adjuvants. Multiple adjuvants are known in the art. Suitable adjuvants include oil emulsions (including oil-in-water water-in-oil, and water-in-oil-in-water emulsion), alum compounds, including both aluminum hydroxide and aluminum phosphate, triterpenoid saponins, immunostimulating complexes, quaternary amine compounds (e.g., Dimethyldioctadecylammonium salts, e.g., bromide), CpG-containing oligonucleotides, oligoribonucleotides, glycolipids such as N-(2-deoxy-2-L-leucylamino-β-D-glucopyranosyl)-N-octadecyldodecanamide also known as BAY®1005 and salts thereof (e.g., the acetate), DEAE Dextran, and combinations thereof.

In certain embodiments, the adjuvant contains a complex formed by Quil A (a triterpenoid saponin) and a sterol (e.g., cholesterol) in an oil-in-water emulsion formed by mineral oil and lecithin known as PREZENT-A®. In other embodiments, the adjuvant comprises a triterpenoid saponin and CpG. In yet other embodiments, the adjuvant comprises a mixture of a CpG oligonucleotide, triterpenoid saponin and the sterol.

Immunogenic compositions and vaccines of the present invention can include one or more veterinarily-acceptable carriers such as solvents, dispersion media, coatings, stabilizing agents, diluents, preservatives, antibacterial and antifungal agents, isotonic agents, adsorption delaying agents, and the like. Diluents can include water, saline, dextrose, ethanol, glycerol, and the like. Isotonic agents can include sodium chloride, dextrose, mannitol, sorbitol, and lactose, among others known to those skilled in the art. Stabilizers include albumin, among others known to the skilled artisan. Preservatives include merthiolate, among others known to the skilled artisan. Antibiotics include, but are not limited to, those from the classes of aminoglycosides, carbapenems, cephalosporins, glycopeptides, macrolides, penicillins, polypeptides, quinolones, sulfonamides, and tetracyclines.

### Forms, Dosages, Routes of Administration

Immunogenic compositions and vaccines of the present invention can be administered to animals to induce an effective immune response against C. chauvoei infection. Accordingly, the present invention provides methods of stimulating an effective immune response against C. chauvoei infection, by administering to an animal a therapeutically effective amount of an immunogenic composition or vaccine of the present invention described herein.

Immunogenic compositions and vaccines of the present invention can be made in various forms depending upon the route of administration. For example, the immunogenic compositions and vaccines can be made in the form of sterile aqueous solutions or dispersions suitable for injectable use. Immunogenic compositions and vaccines can also be made in the form of suspensions or emulsions.

The immunogenic compositions and vaccines generally comprise a veterinarily-acceptable carrier in a volume of from about 0.5 ml to about 5 ml. In another embodiment the volume of the carrier is from about 1 ml to about 4 ml, or from about 2 ml to about 3 ml. In another embodiment, the volume of the carrier is about 1 ml, or is about 2 ml, or is about 5 ml. Veterinarily-acceptable carriers suitable for use in immunogenic compositions and vaccines can be any of those described hereinabove.

In accordance with the methods of the present invention, a single dose can be administered to animals, or, alternatively, two or more inoculations can take place with intervals of from about two to about 26 weeks, e.g., 3 weeks, 4, weeks, 5 weeks, 6 weeks 10 weeks 15 weeks, 20 weeks 25 weeks. Boosting regimens can be required and the dosage regimen can be adjusted to provide optimal immunization. Those skilled in the art can readily determine the optimal administration regimen.

Immunogenic compositions and vaccines can be administered directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which can contain excipients such as salts, carbohydrates and buffering agents (preferably to a pH of from about 3 to about 9, or from about 4 to about 8, or from about 5 to about 7.5, or from about 6 to about 7.5, or about 7 to about 7.5), but, for some applications, they can be more suitably formulated as a sterile non-aqueous solution or as a dried form to be used in conjunction with a suitable vehicle such as sterile, pyrogen-free water.

Formulations for parenteral administration can be formulated to be immediate and/or modified release. Modified release formulations include delayed, sustained, pulsed, controlled, targeted and programmed release. Thus, the immunogenic compositions and/or the vaccines of the invention can be formulated as a solids, semi-solids, or thixotropic liquids for administration as an implanted depot providing modified release of the active compound. Examples of such formulations include drug-coated stents and poly(dl-lactic-coglycolic) acid (PGLA) microspheres.

The following examples are presented as illustrative embodiments, but should not be taken as limiting the scope of the invention. Many changes, variations, modifications, and other uses and applications of this invention will be apparent to those skilled in the art.

### EXAMPLES

### Example 1: Clostridium chauvoei Production Method for Flagella and ccctA Antigens

Quadruple strength media for seed scale and antigen production was prepared as follows. Soy Peptone 200 g/L and Yeast Extract 20 g/L were added to distilled water at 50°+5° C. and mixed until dissolved. A 400 g/L solution of magnesium sulphate was prepared, and sufficient volume added to the dissolved peptone and yeast extract solution to give a concentration of 2.0 g/L magnesium sulphate. The quadruple strength medium is made up to single strength with distilled water. The pH was adjusted to 7.4-7.5 with sodium hydroxide solution. The medium was sterilised by heating to a minimum of 121° C. for a minimum of 30 minutes.

To prepare the final medium (PYE), a sterile solution 10% cysteine HCL and 5% ascorbic acid was added to the base medium to give final concentrations of 0.05% cysteine HCl and 0.025% ascorbic acid and sufficient 50% sterile glucose solution to give a final glucose concentration of 0.2%.

A freeze-dried ampoule of Clostridium chauvoei (strain CH3) was resuspended in sterile Robertson's Cooked Meat Medium, and used to inoculate test tubes containing Robertson's Cooked Meat Medium supplemented with 0.05% cysteine HCl, 0.025% ascorbic acid and 0.2% glucose. The seed stage 1 culture was incubated until heavily turbid and inoculated with 2% v/v inoculum into PYE medium (seed stage 2). Seed stage 2 was incubated until heavily turbid. A fermenter was then inoculated with 5% v/v of culture from seed stage 2 culture. The temperature was held at 37° C. The pH was allowed to drop to pH 6.5, and the controller reset to pH 6.8. Following reset of the pH, 50% glucose feed was added for the next 5 hours at a rate of 7.2 mL/L culture/hr. The culture was complete when the glucose level was zero or when the culture reached stationary phase. The culture was inactivated by the addition of 0.8% v/v formalin. The pH was adjusted to 6.8-6.9 and the formolised culture incubated at 37° C. for 7 days. The inactivated culture was held at 2-8° C. until further processing.

Another preparation of vaccine was manufactured as described above, but using 200 g/L meat peptone instead of 200 g/L soy peptone.

### Example 2: Down Stream Processing of C. chauvoei Antigens

Inactivated fermentation culture (prepared as described above in Example 1) was treated for each indicated group as follows. These processed C. chauvoei antigens were employed in Example 3 below.
T01: (Whole Bulk) Not processed.
T02: (15× MF concentrated) Inactivated culture concentrated 30× via tangential flow filtration via a 0.2 μm molecular weight cutoff membrane cassettes. The cell concentrate was recovered from the system with a saline wash leading to a final volume reduction equal to 15×.
T03: (15×MF concentrated and diafiltered) Inactivated culture concentrated 30× via tangential flow filtration via a 0.2 μm molecular weight cutoff membrane cassettes. The cell concentrate was diafiltered with saline (4× constant volume wash) and the diafiltered cell concentrate recovered from the system with a saline wash leading to a final volume reduction equal to 15×.
T04: (15×UF Cell-free permeate) Permeate collected during the cell concentration was concentrated 30-fold via a tangential flow filtration using 10 kDa molecular weight cut-off membrane cassettes. The concentrated permeate was filter sterilized through a 0.2 μm filter. The permeate concentrate was recovered from the system with a saline wash leading to a final volume reduction equal to 15×.
T05: (15×UF Cell-free permeate diafiltered) Permeate collected during the cell concentration was concentrated 30-fold via a tangential flow filtration using 10 kDa molecular weight cut-off membrane cassettes. The concentrated permeate was diafiltered with saline (4× constant volume wash) and filter sterilized through a 0.2 μm filter. The permeate concentrate was recovered from the system with a saline wash leading to a final volume reduction equal to 15×.
   Analytical Testing of C. chauvoei Downstream Processed Antigens

The downstream processed Clostridium chauvoei antigens (prepared as described above) were tested for ccctA antigen and flagella. The results are presented below in Table 1.

TABLE 1
Flagella and ccctA levels for different Downstream
Processing Treatments for Antigen Lots 065 and 370

| | Flagella | | cctA | |
|---|---|---|---|---|
| | Lot 370 (meat | Lot 065 (soy | Lot 370 (meat | Lot 065 (soy |
| | peptone | peptone | peptone | peptone |
| Treatment | grown) | grown) | grown) | grown) |
| T01 | 0.78 | NT | 9.4 | 39 |
| T02 | 14.2 | NT | 41.7 | NT |
| T03 | 9.8 | 20 | 10 | 131 |
| T04 | 0.37 | NT | 60.3 | 1465 |
| T05 | 0.34 | NT | 93 | 1537 |

### Example 3: Preparation of Experimental Clostridial Vaccine Formulations

All experimental Monovalent Clostridial chauvoei vaccines were formulated with a final concentration of 1.6 mg/mL aluminium hydroxide (as aluminium). Details are given in Table 2 for Lot 370 C. chauvoei antigen.

TABLE 2
Monovalent C chauvoei Vaccine Formulations Formulated With Lot 370 Antigen Component

| | VC1548-1 | VC1548-2 | VC1548-3 | VC1548-4 | VC1548-5 | VC1548-6 | VC1548-7 |
|---|---|---|---|---|---|---|---|
| 3% | 533.3 | 533.3 | 533.3 | 533.3 | 533.3 | 533.3 | 533.3 |
| Aluminium | | | | | | | |
| hydroxide gel | | | | | | | |
| (mL) | | | | | | | |
| C. chauvoei | 0.0001 | 0.0005 | 0.0027 | 0.0108 | 0.0674 | 0.0294 | 0.0289 |
| flagella (Ru) | | | | | | | |
| T03 | | | | | | | |
| C. chauvoei | 0.053 | 0.266 | 1.328 | 5.300 | 33.200 | 0.295 | 0.029 |
| cctA (μg/mL) | | | | | | | |
| T05 | | | | | | | |
| 1% thiomersal | 10.0 | 10.0 | 10.0 | 10.0 | 10.0 | 10.0 | 10.0 |
| solution (mL) | | | | | | | |
| Final Volume | 1000 | 1000 | 1000 | 1000 | 1000 | 1000 | 1000 |

TABLE 3
ULTRAVAC7 In 1 ®Vaccine Formulations Formulated With Lot 370 Antigen

| | Vaccine Formulations | | | | | | | |
|---|---|---|---|---|---|---|---|---|
| Component | VC1574-1 | VC1574-2 | VC1574-3 | VC1574-4 | VC1574-5 | VC1574-6 | VC1574-7 | VC1574-8 |
| 7.7% | 206.7 | 206.7 | 206.7 | 206.7 | 206.7 | 206.7 | 206.7 | 206.7 |
| Aluminium | | | | | | | | |
| hydroxide gel | | | | | | | | |
| (mL) | | | | | | | | |
| C. chauvoei | 0.234 | 0.117 | 0.128 | 0.171 | 0.063 | 0.011 | 0.054 | 0.090 |
| flagella (Ru) | | | | | | | | |
| T03 | | | | | | | | |
| C. chauvoei | 0.239 | 0.120 | 5.420 | 26.680 | 2.360 | 5.300 | 26.560 | 44.230 |
| ccctA (μg/mL) | | | | | | | | |
| T05 | | | | | | | | |
| C. septicum | 13.7 | 13.7 | 13.7 | 13.7 | 13.7 | 13.7 | 13.7 | 13.7 |
| toxoid | | | | | | | | |
| concentrate | | | | | | | | |
| (mL) | | | | | | | | |
| C. tetani | 1.4 | 1.4 | 1.4 | 1.4 | 1.4 | 1.4 | 1.4 | 1.4 |
| toxoid | | | | | | | | |
| concentrate | | | | | | | | |
| (mL) | | | | | | | | |
| C. novyi toxoid | 11.5 | 11.5 | 11.5 | 11.5 | 11.5 | 11.5 | 11.5 | 11.5 |
| concentrate | | | | | | | | |
| (mL) | | | | | | | | |
| C. perfringens | 16.1 | 16.1 | 16.1 | 16.1 | 16.1 | 16.1 | 16.1 | 16.1 |
| type D toxoid | | | | | | | | |
| concentrate | | | | | | | | |
| (mL) | | | | | | | | |
| Leptospira | 477.3 | 477.3 | 477.3 | 477.3 | 477.3 | 477.3 | 477.3 | 477.3 |
| hardjo/Pomona | | | | | | | | |
| Pool (mL) | | | | | | | | |
| 1% thiomersal | 9.2 | 9.2 | 9.2 | 9.2 | 9.2 | 9.2 | 9.2 | 9.2 |
| solution (mL) | | | | | | | | |
| Final Volume | 1000 | 1000 | 1000 | 1000 | 1000 | 1000 | 1000 | 1000 |

All experimental Multivalent Clostridial vaccines (ULTRAVAC® 7 In 1) were formulated with a final concentration of 1.6 mg/mL aluminium hydroxide (as aluminium). Details are given in Table 4 for Lot 065 antigen.

TABLE 4
ULTRAVAC 7 In 1 ®Vaccine Formulations Formulated With Lot 065 Antigen

| | Vaccine Formulations | | | | | | | |
|---|---|---|---|---|---|---|---|---|
| Component | VC1585-1 | VC1585-2 | VC1585-3 | VC1585-4 | VC1585-5 | VC1585-6 | VC15857 | VC1585-8 |
| 4.3% | 372 | 372 | 372 | 372 | 372 | 372 | 372 | 372 |
| Aluminium | | | | | | | | |
| hydroxide gel | | | | | | | | |
| (mL) | | | | | | | | |
| C. chauvoei | 0.19 | 0.119 | 0.059 | 0.059 | 0.059 | 0.03 | 0 | 0.059 |
| flagella (Ru) | | | | | | | | |
| T03 | | | | | | | | |
| C. chauvoei | 18.96 | 0.78 | 0.39 | 0.99 | 3.37 | 3.18 | 2.98 | 23.15 |
| ccctA (ug/mL) | | | | | | | | |
| T05 | | | | | | | | |
| C. septicum | 13.7 | 13.7 | 13.7 | 13.7 | 13.7 | 13.7 | 13.7 | 13.7 |
| toxoid | | | | | | | | |
| concentrate | | | | | | | | |
| (mL) | | | | | | | | |
| C. tetani | 1.4 | 1.4 | 1.4 | 1.4 | 1.4 | 1.4 | 1.4 | 1.4 |
| toxoid | | | | | | | | |
| concentrate | | | | | | | | |
| (mL) | | | | | | | | |
| C. novyi toxoid | 11.7 | 11.7 | 11.7 | 11.7 | 11.7 | 11.7 | 11.7 | 11.7 |
| concentrate | | | | | | | | |
| (mL) | | | | | | | | |
| C. perfringens | 16.3 | 16.3 | 16.3 | 16.3 | 16.3 | 16.3 | 16.3 | 16.3 |
| type D toxoid | | | | | | | | |
| concentrate | | | | | | | | |
| (mL) | | | | | | | | |
| Leptospira | 225.3 | 225.3 | 225.3 | 225.3 | 225.3 | 225.3 | 225.3 | 225.3 |
| hardjo (mL) | | | | | | | | |
| Leptospira | 252.0 | 252.0 | 252.0 | 252.0 | 252.0 | 252.0 | 252.0 | 252.0 |
| Pomona (mL) | | | | | | | | |
| 1% thiomersal | 9.2 | 9.2 | 9.2 | 9.2 | 9.2 | 9.2 | 9.2 | 9.2 |
| solution (mL) | | | | | | | | |
| Final Volume | 1000 | 1000 | 1000 | 1000 | 1000 | 1000 | 1000 | 1000 |

### Example 4: Guinea Pig Protection

For each set of vaccines, guinea pigs were divided randomly into groups of 5 animals. One group of five guinea pigs per set of vaccines was left as control. In the vaccinated groups, each animal received one dose of the test vaccine (Monovalent Vaccine 1 mL dose and ULTRAVAC® 7 In 1 vaccines 1.25 mL dose) subcutaneously in the flank. Twenty-eight days after the first injection, each animal in the test groups received a second injection with the test vaccine subcutaneously in the opposite flank. Fourteen days following the second injection, vaccinated and control animals were challenged with a suspension of viable Clostridium chauvoei spores strain CH4 (0.5 ml) in 5% CaCl₂) solution, injected intramuscularly into the thigh muscle of the right hind leg.

The challenged guinea-pigs were inspected daily for the following 5 days, recording on the worksheet any deaths which occurred. Where a death occurred, obvious symptoms of blackleg edema must be present. At the end of the 5th day challenge, all surviving animal were euthanized. For ethical reasons, moribund animals were euthanized and considered to have died from C. chauvoei infection.

In a first experiment monovalent C. chauvoei vaccines containing different proportions of flagella and cctA were potency tested via virulent challenge in guinea pigs. By increasing the level of cctA in the vaccine in the absence of cells 100% protection was attained (see Table 5). Interestingly, by the addition of cells (flagella) the level of cctA to attain full protection was markedly reduced.

In a second experiment, multivalent ULTRAVAC® 7 In 1 vaccines containing 5 clostridial antigens and two Leptospira antigens were potency tested via virulent challenge in guinea pigs. Full protection was attained with a balance of flagella and cctA. The levels of cctA and flagella required to provide full protection were higher than required for the monovalent vaccine.

In the third experiment, multivalent ULTRAVAC® 7 In 1 vaccines containing 5 clostridial antigens and two Leptospira antigens were potency tested via virulent challenge in guinea pigs. The difference with experiment 2, was that the C. chauvoei antigen used in this experiment was derived from soy peptone grown antigen. Again, full protection was attained with a balance of flagella and cctA antigens.

TABLE 5
Monovalent C chauvoei Vaccine Formulations
Formulated With Lot 370 Antigen

| Vaccine | Flagella, RU | cctA μg/mL | Potency (Survivors) |
|---|---|---|---|
| VC1548-1 | 0.0001 | 0.053 | 0% |
| VC1548-2 | 0.0005 | 0.266 | 20% |
| VC1548-3 | 0.0027 | 1.328 | 20% |
| VC1548-4 | 0.0108 | 5.3 | 80% |
| VC1548-5 | 0.0674 | 33.2 | 100% |
| VC1548-6 | 0.0294 | 0.295 | 100% |
| VC1548-7 | 0.0289 | 0.029 | 100% |

TABLE 6
ULTRAVAC ® 7 In 1 ® Vaccine Formulations
Formulated With Lot 370 Antigen

| Vaccine | Flagella, RU | cctA μg/mL | Potency (Survivors) |
|---|---|---|---|
| VC1574-1 | 0.234 | 0.239 | 40% |
| VC1574-2 | 0.117 | 0.12 | 60% |
| VC1574-3 | 0.128 | 5.42 | 100% |
| VC1574-4 | 0.171 | 26.68 | 100% |
| VC1574-5 | 0.063 | 2.36 | 0% |
| VC1574-6 | 0.011 | 5.3 | 60% |
| VC1574-7 | 0.054 | 26.56 | 80% |
| VC1574-8 | 0.090 | 44.23 | 80% |

TABLE 7
ULTRAVAC ® 7 In 1Vaccine Formulations
Formulated With Lot 065 Antigen

| Vaccine | Flagella RU | cctA μg/mL | Potency (% survivors) |
|---|---|---|---|
| VC1585-1 | 0.119 | 18.96 | 100% |
| VC1585-2 | 0.119 | 0.78 | 60% |
| VC1585-3 | 0.059 | 0.39 | 0% |
| VC1585-4 | 0.059 | 0.99 | 40% |
| VC1585-5 | 0.059 | 3.37 | 100% |
| VC1585-6 | 0.03 | 3.18 | 0% |
| VC1585-7 | 0 | 2.98 | 0% |
| VC1585-8 | 0.059 | 23.15 | 100% |

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

| | Sequence Listing | | |
|---|---|---|---|
| 1 | Information | | |
| 1-1 | File Name | ZP000393-PCT.xml | |
| 1-2 | DTD Version | V1_3 | |
| 1-3 | Software Name | WIPO Sequence | |
| 1-4 | Software Version | 2.0.0 | |
| 1-5 | Production Date | 2022-07-14 | |
| 1-6 | Original free text | | |
| | language code | | |
| 1-7 | Non English free | | |
| | text language | | |
| | code | | |
| | General | | |
| 2 | Information | | |
| 2-1 | Current | | |
| | application: IP | | |
| | Office | | |
| | Current | | |
| | application: | | |
| 2-2 | Application | | |
| | number | | |
| | Current | | |
| | application: | | |
| 2-3 | Filing date | | |
| | Current | | |
| 2-4 | application: | ZP000393A | |
| | Applicant file | | |
| | reference | | |
| 2-5 | Earliest priority | US | |
| | application: IP | | |
| | Office | | |
| 2-6 | Earliest priority | 63/230176 | |
| | application: | | |
| | Application | | |
| | number | | |
| 2-7 | Earliest priority | 2021-08-06 | |
| | application: | | |
| | Filing date | | |
| 2-8en | Applicant name | Zoetis Services LLC | |
| 2-8 | Applicant name: | | |
| | Name Latin | | |
| 2-9en | Inventor name | Anthony Cameron | |
| 2-9 | Inventor name: | | |
| | Name Latin | | |
| 2-10en | Invention title | Clostridium Chauvoei vaccine and method of making | |
| 2-11 | Sequence Total | 1 | |
| | Quantity | | |
| 3-1 | Sequences | | |
| 3-1-1 | Sequence | 1 | |
| | Number [ID] | | |
| 3-1-2 | Molecule Type | AA | |
| 3-1-3 | Length | 317 | |
| 3-1-4- | Features | source | |
| 1 | Location/Qualifiers | 1..317 mol_type = protein | |
| | NonEnglishQualifier | organism = Clostridium Chauvoei | |
| | Value | | |
| 3-1-5 | Residues | MIKRILMLAL ATTTIFSLTL PFSYKAVQAQ ENTCIVETPS | 40 |
| | | EGVKTFTSSD TAYADYNCFK TNLSVTFIED QHNNQLTALV | 80 |
| | | STEGSFIPSG LSRVGGYYQA DMYWPSKYYT TLTTYDRNNR | 120 |
| | | VKITKSIPTN QIDTVSVSET MGYSIGGSLS IEYGKEGPKA | 160 |
| | | GGGINGSYTA QRSVTYDQPD YRTLLMKDSV NSASWEVAFN | 200 |
| | | ATKDGYDRDS YHGIYGNQLF MRYRLYNTGI NNLTTDNNLS | 240 |
| | | SLIVGGFSPK VVIALTAPKG TEESTVKVEY NRFNDQYRLR | 280 |
| | | WSGTEWYGEN NRNSRIDSSS ESFILNWKNH TVEHAGY | 317 |

1. A vaccine against Clostridium chauvoei, said vaccine comprising a C. chauvoei component and additional cctA protein.

2. The vaccine according to claim 1, wherein one dose of said vaccine contains at least about 3.5 μg of said additional cctA protein.

3. The vaccine of claim 1, wherein one dose contains 0.025 to 0.148 RU of flagella.

4. The vaccine of claim 1, wherein one dose contains about 0.074 RU of said flagella.

5. The vaccine of claim 4, wherein one dose contains about 5 μg of said additional cctA protein.

6. (canceled)

7. (canceled)

8. The vaccine according to claim 1, wherein said C. chauvoei component is a bacterin, a whole cell extract, a partial cell extract, or a combination thereof.

9. The vaccine according to claim 8, wherein C. chauvoei component is said partial cell membrane that comprises a membrane fraction.

10. A method of preparing a vaccine against C. chauvoei infection, said method comprising:
a. Culturing C. chauvoei;
b. Collecting culture media;
c. Concentrating C. chauvoei from said culture media;
d. Concentrating cctA from said culture media; and
e. Combining said concentrated cctA with C. chauvoei.

11. The method of claim 10 comprising a step of microfiltrating said culture media to obtain a microfiltered retentate and a microfiltered permeate.

12. The method of claim 10, wherein the step of culturing C. chauvoei is performed in a culture media comprising a peptone of plant origin, a peptone of animal origin, or a combination thereof.

13. The method of claim 11, wherein the step of concentrating said cctA from said culture media comprises:
a. Ultrafiltration of said microfiltered permeate, to obtain an ultrafiltered retentate, followed by
b. Diafiltration, to obtain a diafiltered retentate, followed by
c. Sterile filtration, to obtain a sterile solution of said additional cctA protein.

14. The method of claim 13 wherein said ultrafiltration comprises passing said microfiltered permeate through a 10 kDa cassette.

15. The method of claim 13, wherein said diafiltration comprises passing said ultrafiltered retentate through a 10 kDa cassette.

16. The method of claim 15, wherein said diafiltration is followed by a sterile filtration of the diafiltered retentate through a 0.2 μm filter to prepare the sterile solution of additional cctA protein.

17. The method of claim 11, wherein the microfiltered retentate is diafiltered to obtain the C. chauvoei component.

18. The method according to claim 10 wherein at least a portion of the C. chauvoei component according to claim 17 and at least a portion of the sterile solution of additional cctA protein are combined.

19.-22. (canceled)

23. The vaccine according to claim 1 further comprising at least one additional antigen selected from the group consisting of Clostridium septicum, Clostridium haemolyticum, Clostridium novyi, Clostridium sordellii, Clostridium tetani, Clostridium perfringens types C and D, Erysipelothrix rhusiopathiae, Leptospira borgpetersenii serovar Hardjo, Leptospira interrogans serovar Pomona and Mannheimia haemolytica antigens.

24. (canceled)

25. The vaccine according to claim 1, further comprising an adjuvant.

26. A method of preventing C. chauvoei infection in a subject comprising administering to said subject the vaccine according to claim 1.

27. The method of claim 26 wherein said subject is a bovine or an ovine subject.
